(19) United States
(12) Patent Application Publication
(10) Pub. No.: US 2026/0049266 A1
(43) Pub. Date: Feb. 19, 2026

(54) CARTRIDGE AND SYSTEM

(71) Applicants: CANON MEDICAL SYSTEMS CORPORATION, Otawara-shi (JP); Canon Kabushiki Kaisha, Tokyo (JP); CiRA Foundation, Kyoto-shi (JP)

(72) Inventors: Yosuke YAMAGUCHI, Kawasaki (JP); Kazutaka YANAGITA, Yokohama (JP); Tetsuya SAKAI, Ryugasaki (JP); Hirotoshi TAHARA, Ohta (JP); Tomoko HIRANO, Inagawa (JP)

(73) Assignees: CANON MEDICAL SYSTEMS CORPORATION, Otawara-shi (JP); Canon Kabushiki Kaisha, Tokyo (JP); CiRA Foundation, Kyoto-shi (JP)

(21) Appl. No.: 19/297,490

(22) Filed: Aug. 12, 2025

(30) Foreign Application Priority Data
Aug. 13, 2024 (JP) 2024-134957

(51) Int. Cl. C12M 3/00; C12M 1/00; C12M 1/34; C12M 1/36

(52) U.S. Cl. CPC C12M 23/42; C12M 23/40; C12M 23/48; C12M 41/06; C12M 41/32; C12M 41/48

(57) ABSTRACT

A cartridge according to the embodiment is a cartridge in which performs processing of a cell is performed and includes a channel through which a reagent used in the processing flows, a connection configured to connect a reagent container containing the reagent to the channel, and an opening/closing portion for connecting the reagent container to the connection and placing the reagent container in the cartridge.

### CROSS-REFERENCE TO RELATED APPLICATIONS

This application is based upon and claims the benefit of priority from Japanese Patent Application No. 2024-134957, filed on Aug. 13, 2024; the entire contents of which are incorporated herein by reference.

### FIELD

Embodiments described herein relate generally to a cartridge and a system.

### BACKGROUND

Conventionally, induced pluripotent stem cells (hereafter referred to as iPS cells) are produced manually by skilled operators in equipment, such as biosafety cabinets. Specifically, devices that produce iPS cells are known, including closed cell production devices designed for a predetermined automated process, such as cell culture, and cell processing devices that allow operators to organize any desired processes.

### BRIEF DESCRIPTION OF THE DRAWINGS

FIG. 1 is a perspective view of an example of the configuration of a cartridge according to an embodiment;

FIG. 2 is a side view of an example of the configuration of the cartridge according to the embodiment;

FIG. 3 is a block diagram of an example of the functional configuration of a cell production system according to the embodiment;

FIG. 4 is a schematic for explaining an example of the procedure of placing a reagent container in the cartridge according to the embodiment;

FIG. 5 is a table indicating an example of identification information of the cartridge according to the embodiment;

FIG. 6 is a schematic of an example of the configuration of a first outer cover according to a first modification;

FIG. 7 is a schematic of an example of the configuration of an opening/closing portion according to a second modification;

FIG. 8 is a schematic of another example of the configuration of the opening/closing portion according to the second modification;

FIG. 9 is a table indicating an example of the identification information according to a third modification; and

FIG. 10 is a schematic of an example of display information output by a display unit according to the third modification.

### DETAILED DESCRIPTION

A cartridge according to the embodiment is a cartridge in which processing of a cell is performed. The cartridge includes a channel through which a reagent used in the processing flows, a connection configured to connect a reagent container containing the reagent to the channel, and an opening/closing portion for connecting the reagent container to the connection and placing the reagent container in the cartridge.

Exemplary embodiments of a cartridge and a system are described below in greater detail with reference to the accompanying drawings. The cartridge and the system according to the present disclosure are not limited by the embodiments described below. The embodiments can be combined with other embodiments and conventional techniques as long as there is no contradiction in the contents. In the following description, like components are denoted by like reference numerals, and duplicated explanation thereof is omitted.

A cell production system according to the present embodiment includes a double-structured container (cartridge) including a housing container that houses a closed flow path, and a cell production device on which the cartridge is mounted. The cartridge is a sample processing cartridge, for example. The cartridge is a container in which the processing of a cell is performed. The cell processing performed in the cartridge may include blood cell separation, expansion culture, factor introduction, planar culture, and collection, which will be described later.

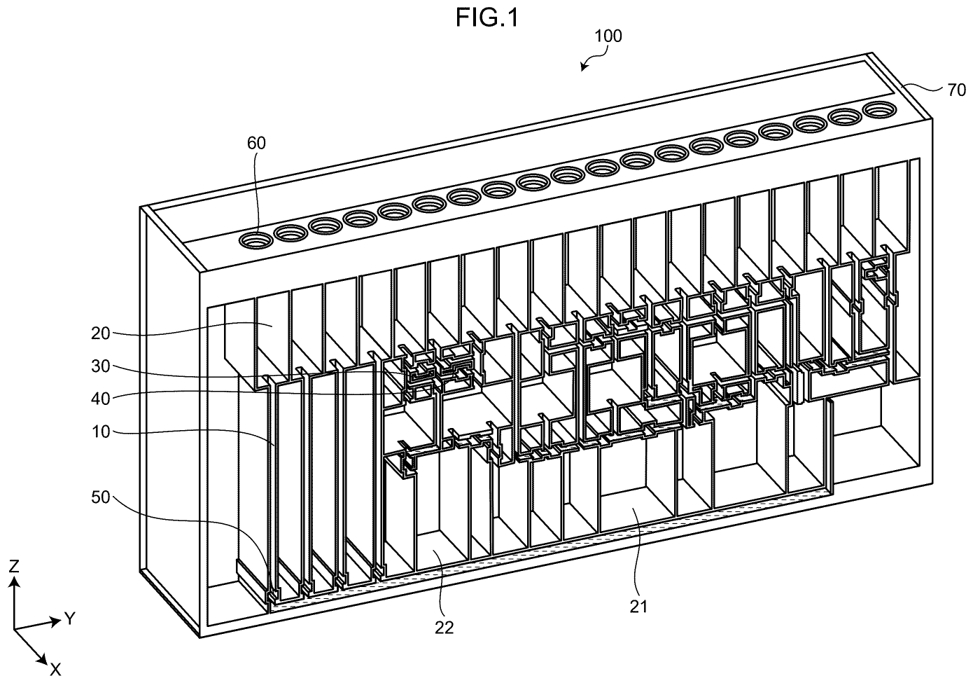

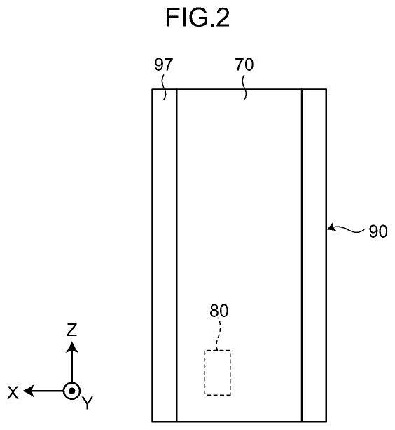

A cell production system according to the present embodiment is described below in greater detail with reference to examples. FIG. 1 is a perspective view of an example of the configuration of a cartridge 100 in the cell production system according to the present embodiment. FIG. 2 is a side view of an example of the configuration of the cartridge 100. In the present embodiment, the axial direction or the vertical direction perpendicular to the bottom surface of the cartridge 100 is defined as a Z-axis direction, and two directions orthogonal to the Z-axis direction and to each other are defined as an X-axis direction and a Y-axis direction.

As illustrated in FIGS. 1 and 2, the cartridge 100 includes a housing container 70, a door opening/closing unit 80, a first outer cover 90, and a second outer cover 97. First, the components housed in the housing container 70 are described. While FIG. 1 illustrates only the housing container 70, the positions with respective reference numerals, which will be described later, are provided with the corresponding units when the cartridge 100 is assembled. The reference numerals in FIG. 1 indicate representative placement positions for the respective units, and the positions not with reference numerals are also provided with units.

The housing container 70 houses a connection channel 10, a liquid feeding vessel 20, a culture vessel 21, a waste liquid vessel 22, a filter 30, a microchannel 40, and valves 50. The housing container 70 also has pressurization/decompression ports 60. The entire flow path composed of the connection channel 10, the liquid feeding vessel 20, the culture vessel 21, the waste liquid vessel 22, the filter 30, the microchannel 40, and other components forms a closed system to prevent bacteria or the like from entering from the outside. The closed system herein refers to a space having no points communicating with the outside or a space in which all the points communicating with the outside aseptically communicate by a sterile filter or the like. The entire flow path forming the closed system is hereinafter referred to as a closed flow path.

The connection channel 10 is a channel through which a reagent used for the processing of a cell flows. The connection channel 10 is also referred to simply as a channel. The connection channel 10 is a pipe for feeding a reagent, such as a culture medium, used for cell culture, and a cell suspension containing subject cells to be cultured, for example. The connection channel 10 is composed of, for example, a tube forming a non-branching portion of the channel and a three-way joint forming a branching portion of the channel.

The tube serving as the non-branching portion is made of flexible material, such as silicone and resin, and the three-way joint serving as the branching portion is made of relatively hard resin, for example. When a cell production device connected to the cartridge 100 applies pressure to the liquid feeding vessel 20 via the pressurization/decompression port 60, for example, a liquid, such as the reagent and the cell suspension, accumulated in the liquid feeding vessel 20 flows through the connection channel 10 to the culture vessel 21.

The liquid feeding vessel 20 is a vessel that temporarily stores therein various liquids, such as biological samples and reagents. The liquid feeding vessel 20 is made of hard resin, for example. The liquid feeding vessel 20 is connected to the connection channel 10 to introduce and discharge a liquid and to the pressurization/decompression port 60 to receive pressure.

For example, when the liquid feeding vessel 20 temporarily stores therein a liquid, the cell production device applies pressure to the pressurization/decompression port 60, thereby causing the liquid stored in the liquid feeding vessel 20 to flow into the connection channel 10.

The culture vessel 21 is a vessel for culturing cells to be cultured inside it. The culture vessel 21 includes a culture dish, a supply tube, a waste liquid tube, and a collection tube, for example. The culture dish is a dish for holding cells to be cultured and a culture medium used for culture. The supply tube is a tube for supplying a reagent, such as a culture medium, to the culture dish. The waste liquid tube is a tube for discharging the liquid in the culture dish and flowing the liquid into the waste liquid vessel 22 to change the culture medium. The collection tube is a tube for collecting the cultured cells and flowing them into a vessel at a subsequent stage.

The waste liquid vessel 22 stores therein a waste liquid generated in a series of cell culture and transmitted from the connection channel 10.

The filter 30 captures necessary components and removes unnecessary components from a biological sample. For example, the filter 30 allows blood cells smaller than a predetermined size to pass therethrough as the unnecessary components and captures blood cells equal to or larger than the predetermined size as the necessary components.

The microchannel 40 classifies subject cells from the cell suspension from which the blood cells captured by the filter 30 are collected. For example, the microchannel 40 classifies nucleated cells from the cell suspension. The microchannel 40 classifies nucleated cells from the cell suspension using a known method of separating blood cells of different sizes in the cross section of the channel by advection due to Dean vortices generated in the helical microchannel 40 and by lift force caused by velocity distribution on the channel wall surface, for example.

The valve 50 is a unit that switches the connection channel 10 between a state where no liquid can pass through the connection channel 10 (closed state) and a state where a liquid can pass through the connection channel 10 (open state). With a plurality of valves 50 and the branching portion (e.g., a three-way joint) of the connection channel 10, the channel can be switched by operating the valves 50.

The valve 50 includes a fixed magnetic plate and a magnet that can move closer to and away from the magnetic plate, for example. The tube of the connection channel 10 is provided between the magnetic plate and the magnet. With this configuration, when no magnetic force is applied from the outside of the cartridge 100, the magnet and the magnetic plate come closer to each other by attraction and flatten the tube of the connection channel 10, thereby bringing the connection channel 10 into the closed state.

By contrast, another magnet (hereinafter referred to as “opening/closing magnet”) is brought closer to the magnet of the valve 50 in the cartridge 100 from the opposite side of the magnetic plate outside the cartridge 100. As a result, the magnet of the valve 50 moves away from the magnetic plate, thereby bringing the connection channel 10 into the open state. The opening/closing magnet is provided to the cell production device together with a drive unit that drives it.

The unit serving as the end of the flow path out of the units connected via the connection channel 10 communicates with the inside or the outside of the cartridge 100 via a sterile filter. The waste liquid vessel 22, for example, communicates with the space inside the cartridge 100 via a sterile filter.

Therefore, the connection channel 10, the liquid feeding vessel 20, the culture vessel 21, the waste liquid vessel 22, the filter 30, the microchannel 40, and the valves 50 constitute one closed system as a whole.

The pressurization/decompression port 60 is a communication port for receiving the application of positive or negative pressure by the cell production device and causes the liquid feeding vessel 20 to communicate with the external space of the cartridge 100 via a sterile filter. Applying the pressure to the liquid feeding vessel 20 via the pressurization/decompression port 60 causes the liquid in the closed flow path to flow. The pressurization/decompression port 60 is formed above the liquid feeding vessel 20 that stores therein biological samples and reagents, for example.

The pressurization/decompression port 60 is connected to a regulator of the cell production device, for example. When the regulator is driven, the liquid, such as biological samples and reagents, stored in the liquid feeding vessel 20 is pushed out by pressure and flows into the connection channel 10 connected to the liquid feeding vessel 20.

The housing container 70 is a container that houses the closed flow path. The housing container 70 has openings. The cartridge 100 is formed by housing the closed flow path in the housing container 70 and closing the openings. The closed flow path and the housing container 70 form a double closed structure. If the closed flow path is damaged, this configuration can prevent leakage of the sample or the like to the outside. The cartridge 100 may be configured as a multi-structure container using the housing container 70 having a double or more structure.

The housing container 70 according to the present embodiment is made of hard resin, metal, or other material and has such a shape that can house the closed flow path, such as a rectangular or cubic shape.

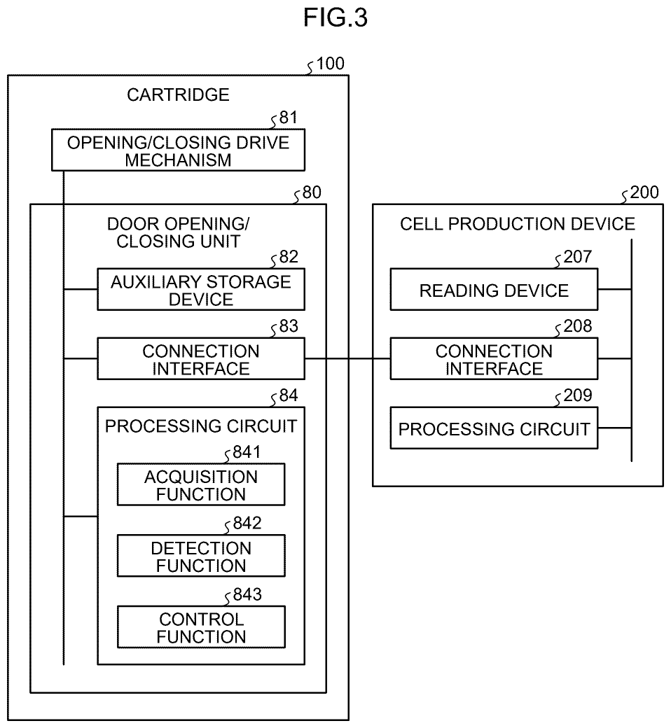

The housing container 70 also includes the door opening/closing unit 80. The door opening/closing unit 80 is provided in the cartridge 100. The following describes the functional configuration of the door opening/closing unit 80 with reference to FIG. 3.

FIG. 3 is a block diagram of an example of the functional configuration of the cell production system according to the present embodiment. The door opening/closing unit 80 includes an auxiliary storage device 82, a connection interface 83, and a processing circuit 84, and is connected to an opening/closing drive mechanism 81. The door opening/closing unit 80 is connected to a cell production device 200 via the connection interface 83.

The cell production device 200 is connected to the door opening/closing unit 80 of the cartridge 100 via a connection interface 208. The cell production device 200 is an example of a cell processing device. The cell production device 200 includes a reading device 207, which will be described later, and a processing circuit 209 that controls the reading device 207 and other units of the cell production device 200. The processing circuit 84 of the door opening/closing unit 80 performs various kinds of processing based on signals from the cell production device 200, for example.

The opening/closing drive mechanism 81 is a mechanism that opens and closes an opening/closing portion included in the first outer cover 90, which will be described later. Specifically, the opening/closing drive mechanism 81 is a mechanism that opens and closes a hinged door of the opening/closing portion included in the first outer cover 90. The opening/closing drive mechanism 81 is a motor, for example. The opening/closing drive mechanism 81 is not limited to a motor and may be any device that opens and closes the hinged door of the opening/closing portion.

The auxiliary storage device 82 is a storage device, such as a hard disk drive (HDD), a solid state drive (SSD), and an integrated circuit storage device, that stores therein various kinds of information. Instead of HDD, SSD, or the like, the auxiliary storage device 82 may be a storage device that reads and writes various kinds of information from and to a portable storage medium, such as a compact disc (CD), a digital versatile disc (DVD), and a flash memory, or a semiconductor memory element, such as a random access memory (RAM).

The connection interface 83 includes a communication interface, for example. The communication interface performs data communications with the cell production device 200, for example. The communication standards between the communication interface and the network may be any desired standards, such as standards for wireless communication. The information holder will be described later in greater detail.

The processing circuit 84 controls the entire operation of the door opening/closing unit 80 according to the information output from the auxiliary storage device 82 or the connection interface 83. For example, the processing circuit 84 includes a processor, such as a central processing unit (CPU), a micro processing unit (MPU), and a graphics processing unit (GPU), and a memory, such as a read only memory (ROM) and a RAM, as hardware resources.

The processing circuit 84 may be implemented by an application specific integrated circuit (ASIC), a field programmable gate array (FPGA), a complex programmable logic device (CPLD), a simple programmable logic device (SPLD), or the like.

The processing circuit 84 includes an acquisition function 841, a detection function 842, and a control function 843, for example. In the acquisition function 841, the detection function 842, and the control function 843, each function is stored in the auxiliary storage device 82 in the form of a computer program executable by a computer. The processing circuit 84 implements the acquisition function 841, the detection function 842, and the control function 843 by a processor that executes the computer program loaded on the memory of the processing circuit 84.

Specifically, the processing circuit 84 corresponds to a processor that reads a computer program from the auxiliary storage device 82 and executes the computer program to implement the function corresponding to the computer program. In other words, the processing circuit 84 that has read a computer program has a function corresponding to the read computer program. Each function of the detection function 842 and the control function 843 is not necessarily implemented by a single processing circuit. The processing circuit may be configured by combining a plurality of independent processors, and the processors may execute computer programs to implement the respective functions of the acquisition function 841, the detection function 842, and the control function 843. The processing circuit 84 that implements the acquisition function 841, the detection function 842, and the control function 843 is an example of an acquirer, a detector, and a controller.

The processing circuit 84 acquires identification information output from the connection interface 83 by the acquisition function 841. The acquisition function 841 stores the acquired identification information in the auxiliary storage device 82. The identification information will be described later in greater detail.

The processing circuit 84 detects that the opening/closing portion has been opened by the detection function 842. The detection function 842 stores opening information indicating that the opening/closing portion has been opened in the auxiliary storage device 82. The opening/closing portion will be described later in greater detail.

The processing circuit 84 collectively controls the door opening/closing unit 80 by the control function 843. For example, the control function 843 controls the opening/closing drive mechanism 81 according to the identification information. When the reading device 207 reads the identification information stored in the information holder, for example, the control function 843 determines the opening/closing portion to be opened out of a plurality of opening/closing portions based on the identification information of a biological sample container or a reagent container and controls the opening/closing drive mechanism 81.

Referring back to FIG. 2, the openings of the housing container 70 are covered by the first outer cover 90 and the second outer cover 97. To prevent leakage of the reagent or the sample inside the housing container 70 and intrusion of foreign matter from the outside of the housing container 70, the part (frame) of the first outer cover 90 and the second outer cover 97 that comes into contact with the housing container 70 is preferably provided with packing or the like.

The housing container 70 may have a double (multiple) structure for each section. To culture iPS cells, for example, the housing container 70 may have a double structure for each of a section for feeding a reagent, such as a culture medium, a section for blood cell separation, a section for expansion culture, a section for factor introduction, and a section for planar culture and collection.

Next, the first outer cover 90 and the second outer cover 97 are described. The first outer cover 90 and the second outer cover 97 are members that close the opening of the housing container 70. The first outer cover 90 and the second outer cover 97 are made of hard resin, for example. The first outer cover 90 and the second outer cover 97 are attached to the housing container 70 to cover the openings of the housing container 70 using screws or other fasteners, for example.

The first outer cover 90 includes a plurality of opening/closing portions, an information holder, a notifier, and a display unit. The opening/closing portions are opening/closing mechanisms that allow the operator to access a connection that connects the biological sample container that stores therein the biological sample or the reagent container that stores therein the reagent. The opening/closing portion is provided to the first outer cover 90 to cover the front surface of a space for placing the container (container placement space) formed near the liquid feeding vessel 20. The opening/closing portion is a member for connecting the reagent container to the connection and placing the reagent container in the cartridge 100. The opening/closing portion includes a hinged door that opens toward the outside of the cartridge 100, for example. Alternatively, the opening/closing portion may have a sliding door structure that slides along the outer surface of the first outer cover 90, for example.

The opening/closing portion includes the opening/closing drive mechanism 81 that opens and closes the opening/closing portion based on the control by the processing circuit 84. Specifically, the opening/closing portion includes the opening/closing drive mechanism 81 that opens and closes the hinged door based on the control by the control function 843 of the processing circuit 84 of the door opening/closing unit 80. The opening/closing portions preferably include packing or the like to bring the hinged door into firmly contact with the opening in the closed state. In other words, the container placement space in the opening/closing portions is preferably sealed in the closed state. With this configuration, a double closed structure including the container placement space can be formed.

The information holder holds the identification information for identifying the cartridge 100. The information holder is provided with a mark obtained by encoding the identification information of the cartridge 100. Examples of the mark include, but are not limited to, a bar code, a one-dimensional pixel code, a two-dimensional pixel code, etc. The information holder is composed of a radio frequency identification (RFID) tag, for example. In other words, the RFID tag holds the identification information for identifying the cartridge 100.

The notifier notifies the operator of various kinds of information output by the control function 843 of the processing circuit 84. The notifier is a speaker, for example.

The display unit displays various kinds of information output by the control function 843 of the processing circuit 84. The display unit is a display, for example. The display may be a liquid crystal display (LCD), an organic electro luminescence display (OELD), or other desired displays, for example.

The following describes the procedure in which the operator connects the reagent container to the connection channel 10 and places the reagent container in the cartridge 100.

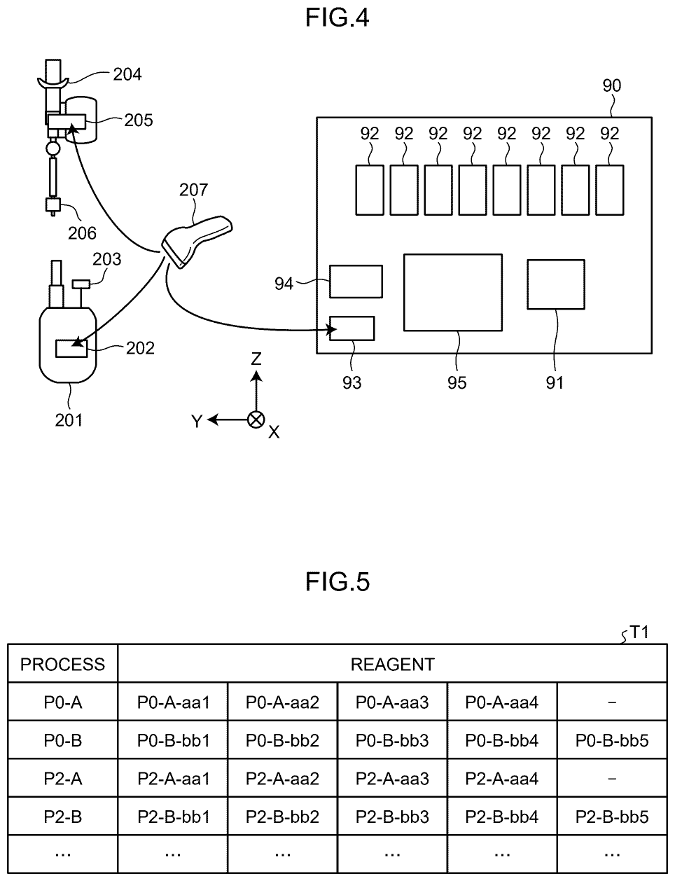

FIG. 4 is a schematic for explaining an example of the procedure of placing the reagent container in the cartridge 100 according to the embodiment. FIG. 4 illustrates the first outer cover 90 when the cartridge 100 is viewed from the front. FIG. 4 also illustrates a plurality of opening/closing portions 91 and 92 included in the first outer cover 90, and an information holder 93, a notifier 94, and a display unit 95 of the cartridge 100. The opening/closing portion 91 corresponds to the connection channel 10 where the biological sample container is to be placed, and the opening/closing portion 92 corresponds to the connection channel 10 where the reagent container is to be placed.

A biological sample container 201 illustrated in FIG. 4 includes an information holder 202 and a connection 203. The information holder 202 holds identification information for identifying the biological sample. The connection 203 is connected to the connection channel 10. A reagent container 204 illustrated in FIG. 4 includes an information holder 205 and a connection 206. The information holder 205 holds identification information for identifying the reagent. The connection 206 is connected to the connection channel 10. FIG. 4 also illustrates a reading device 207 that reads the information holder 202 attached to the biological sample container 201 and the information holder 205 attached to the reagent container 204.

The reading device 207 is a bar code reader that can optically read optical marks, for example, and is connected to the cell production device 200. The information holder 93, the information holder 202, and the information holder 205 described above are each provided with an optical mark obtained by encoding the identification information, that is, a bar code, for example. The information holder 93, the information holder 202, and the information holder 205 may be each composed of an RFID tag, for example.

The operator uses the reading device 207 to read the optical mark on the information holder 93 of the cartridge 100, for example. The identification information indicated by the optical mark read by the reading device 207 is transmitted to the connection interface 83 of the door opening/closing unit 80 via the circuit of the cell production device 200. The acquisition function 841 of the door opening/closing unit 80 acquires the identification information via the connection interface 83 and stores the acquired identification information in the auxiliary storage device 82.

The identification information held by the information holder 93 of the cartridge 100 is described with reference to FIG. 5. FIG. 5 is a table indicating an example of the identification information of the cartridge 100 according to the embodiment. Table T1 illustrated in FIG. 5 is information in which a process, such as cell culture, is associated with reagents used in the process. In other words, the identification information held by the information holder 93 of the cartridge 100 is information including process information indicating the process, such as cell culture, and reagent information indicating the reagents used in the process.

In Table T1 illustrated in FIG. 5, each process is associated with the reagent information on a plurality of different reagents because the reagents to be placed in the cartridge 100 differ between the processes. The identification information of the cartridge 100 may include information indicating the biological sample used in the process.

Referring back to FIG. 4, the control function 843 of the door opening/closing unit 80 controls the notifier 94 such that the notifier 94 outputs audio information that corresponds to the identification information of the cartridge 100, based on the identification information. The notifier 94 outputs the audio information. The audio information is information on the process included in the process information, for example. The control function 843 of the door opening/closing unit 80 controls the display unit 95 such that the display unit 95 displays text information that corresponds to the identification information of the cartridge 100, based on the identification information. The display unit 95 displays the text information. The text information is information on the process included in the process information, for example.

Next, the procedure of placing the biological sample container 201 to the connection channel 10 is described. The operator uses the reading device 207 to read the information holder 202 of the biological sample container 201, for example. The reading device 207 outputs the identification information held by the information holder 202 to the connection interface 83 of the door opening/closing unit 80. The acquisition function 841 of the door opening/closing unit 80 acquires the identification information output from the connection interface 83 and stores the acquired identification information in the auxiliary storage device 82.

The control function 843 of the door opening/closing unit 80 controls the notifier 94 such that the notifier 94 outputs audio information that corresponds to the identification information of the biological sample container 201, based on the identification information. The notifier 94 outputs the audio information. The control function 843 of the door opening/closing unit 80 controls the display unit 95 such that the display unit 95 displays text information that corresponds to the identification information of the biological sample container 201, based on the identification information. The notifier 94 displays the text information. The control function 843 of the door opening/closing unit 80 controls the opening/closing drive mechanism 81 of the opening/closing portion 92 based on the identification information of the biological sample container 201 and the identification information of the cartridge 100. If the identification information of the biological sample container 201 is included in the identification information of the cartridge 100, for example, the control function 843 controls the opening/closing drive mechanism 81 of the opening/closing portion 92 such that the opening/closing drive mechanism 81 operates.

If the identification information of the biological sample container 201 is not included in the identification information of the cartridge 100, for example, the control function 843 controls the opening/closing drive mechanism 81 of the opening/closing portion 92 such that the opening/closing drive mechanism 81 does not operate. Furthermore, if the identification information of the biological sample container 201 is included in the identification information of the cartridge 100, but the biological sample container 201 has already been connected to the connection of the connection channel 10, for example, the control function 843 controls the opening/closing drive mechanism 81 of the opening/closing portion 92 such that the opening/closing drive mechanism 81 does not operate.

In other words, the control function 843 determines whether an article is lacking based on the identification information of the biological sample container 201 and the identification information of the cartridge 100. If it is determined that an article is lacking, the control function 843 controls the opening/closing drive mechanism 81 of the opening/closing portion 92 such that the opening/closing drive mechanism 81 operates.

The opening/closing drive mechanism 81 opens the opening/closing portion 91. The detection function 842 of the door opening/closing unit 80 detects that the opening/closing portion 91 has been opened and controls the notifier 94 such that the notifier 94 outputs audio information that corresponds to the opening information indicating that the opening/closing portion 91 has been opened. The notifier 94 outputs the audio information. The operator connects the connection 203 of the biological sample container 201 to the connection of the connection channel 10 for which the opening/closing portion 91 is opened, and closes the opening/closing portion 91.

Next, the procedure of placing the reagent container 204 to the connection channel 10 is described. The operator uses the reading device 207 to read the information holder 205 of the reagent container 204, for example. The reading device 207 outputs the identification information held by the information holder 205 to the connection interface 83 of the door opening/closing unit 80. The acquisition function 841 of the door opening/closing unit 80 acquires the identification information output from the connection interface 83 and stores the acquired identification information in the auxiliary storage device 82.

The control function 843 of the door opening/closing unit 80 controls the notifier 94 such that the notifier 94 outputs audio information that corresponds to the identification information of the reagent container 204, based on the identification information. The notifier 94 outputs the audio information. The control function 843 of the door opening/closing unit 80 controls the display unit 95 such that the display unit 95 displays text information that corresponds to the identification information of the reagent container 204, based on the identification information. The notifier 94 displays the text information.

The control function 843 of the door opening/closing unit 80 determines an opening/closing portion 92 to be opened out of the opening/closing portions 92, based on the identification information of the reagent container 204 and the identification information of the cartridge 100 and controls the opening/closing drive mechanism 81 of the determined opening/closing portion 92. If the identification information of the reagent container 204 is included in the identification information of the cartridge 100, for example, the control function 843 determines the opening/closing portion 92 to be opened out of the opening/closing portions 92 and controls the opening/closing drive mechanism 81 of the opening/closing portion 92 such that the opening/closing drive mechanism 81 operates.

If the identification information of the reagent container 204 is not included in the identification information of the cartridge 100, for example, the control function 843 determines to open none of the opening/closing portions 92 and controls the opening/closing drive mechanisms 81 of the opening/closing portions 92 such that they do not operate. Furthermore, if the identification information of the reagent container 204 is included in the identification information of the cartridge 100, but the reagent container 204 has already been connected to the connection of the connection channel 10, for example, the control function 843 controls the opening/closing drive mechanism 81 of the opening/closing portion 92 such that the opening/closing drive mechanism 81 does not operate.

In other words, the control function 843 determines whether an article is lacking based on the identification information of the reagent container 204 and the identification information of the cartridge 100. If it is determined that an article is lacking, the control function 843 controls the opening/closing drive mechanism 81 of the opening/closing portion 92 such that the opening/closing drive mechanism 81 operates.

The opening/closing drive mechanism 81 opens the opening/closing portion 92. The detection function 842 of the door opening/closing unit 80 detects that the opening/closing portion 92 has been opened and controls the notifier 94 such that the notifier 94 outputs audio information that corresponds to the opening information indicating that the opening/closing portion 92 has been opened. The notifier 94 outputs the audio information. The operator connects the connection 206 of the reagent container 204 to the connection of the connection channel 10 for which the opening/closing portion 92 is opened, and closes the opening/closing portion 92.

When the control function 843 of the door opening/closing unit 80 according to the embodiment described above controls the opening/closing drive mechanism 81 of the opening/closing portion 91 or the opening/closing portion 92 such that the opening/closing drive mechanism 81 does not operate, the control function 843 may output information indicating that the biological sample container 201 or the reagent container 204 is not the one used in the process and/or information indicating some kinds of error to the notifier 94 and the display unit 95.

The method of outputting the information indicating that the biological sample container 201 or the reagent container 204 is not the one used in the process is, for example, to notify the operator of the fact in natural language, such as “this is not the reagent used in the process”, by the notifier 94 and/or to display the fact on the display unit 95. The method of outputting the information indicating some kinds of error is, for example, to output a beep from the notifier 94 and/or to display an error icon on the display unit 95.

In the description above, the control function 843 of the door opening/closing unit 80 controls the opening/closing drive mechanism 81 of the opening/closing portion 91 or the opening/closing portion 92. Alternatively, the control function 843 may be provided to the processing circuit 209 of the cell production device 200, and the opening/closing drive mechanism 81 may be driven based on the control by the processing circuit 209 of the cell production device 200. Furthermore, the acquisition function 841 and the detection function 842 of the door opening/closing unit 80 may also be provided to the processing circuit 209 of the cell production device 200, and the auxiliary storage device 82 may also be provided to the cell production device 200. In this case, the door opening/closing unit 80 does not necessarily include the processing circuit 84.

As described above, the cartridge 100 according to the embodiment is a cartridge in which processing of a cell is performed. The cartridge 100 includes a channel through which a reagent used in the processing flows, a connection that connects a reagent container containing the reagent to the channel, and an opening/closing portion for connecting the reagent container to the connection and placing the reagent container in the cartridge 100.

The cartridge 100 according to the embodiment further includes an information holder that holds identification information for identifying the cartridge 100. The cartridge 100 according to the embodiment further includes a detector that detects that the opening/closing portion has been opened. The cartridge 100 according to the embodiment includes a plurality of opening/closing portions, and the opening/closing portion includes the opening/closing drive mechanism 81 that opens and closes the opening/closing portion based on control by a device on which the cartridge 100 is mounted.

In the cartridge 100 according to the present embodiment, for example, an operator uses the reading device 207 to read an optical mark on the information holder 93 of the cartridge 100. As a result, the acquisition function 841 of the door opening/closing unit 80 or the processing circuit 209 of the cell production device 200 acquires the identification information held by the information holder 93 of the cartridge 100. The identification information includes process information indicating a process, such as cell culture, and reagent information indicating a reagent used in the process.

The operator uses the reading device 207 to read the information holder 202 of the biological sample container 201 or the information holder 205 of the reagent container 204. As a result, the control function 843 of the door opening/closing unit 80 or the processing circuit 209 of the cell production device 200 controls the notifier 94 such that the notifier 94 outputs audio information that corresponds to the identification information of the biological sample container 201 or the reagent container 204, based on the identification information. Thus, the notifier 94 outputs the audio information.

The control function 843 of the door opening/closing unit 80 or the processing circuit 209 of the cell production device 200 controls the display unit 95 such that the display unit 95 displays text information that corresponds to the identification information of the biological sample container 201 or the reagent container 204, based on the identification information. Thus, the notifier 94 displays the text information. Thus, the operator can find out the identification information of the biological sample container 201 or the reagent container 204 to be placed in the cartridge 100.

The control function 843 of the door opening/closing unit 80 or the processing circuit 209 of the cell production device 200 determines an opening/closing portion to be opened out of the opening/closing portions, based on the identification information of the biological sample container 201 or the identification information of the reagent container 204 and on the identification information of the cartridge 100 and controls the opening/closing drive mechanism 81 of the determined opening/closing portion. Thus, the opening/closing drive mechanism 81 opens the opening/closing portion. The detection function 842 of the door opening/closing unit 80 or the processing circuit 209 of the cell production device 200 detects that the opening/closing portion has been opened and controls the notifier 94 such that the notifier 94 outputs audio information that corresponds to the opening information indicating that the opening/closing portion 91 has been opened. Thus, the notifier 94 outputs the audio information.

The operator connects the connection of the biological sample container 201 or the reagent container 204 to the connection of the connection channel 10 through the opened opening/closing portion and closes the opening/closing portion. This mechanism can prevent the operator from erroneously connecting the biological sample container 201 and the reagent container 204.

Therefore, the cartridge 100 according to the embodiment can reduce the complexity of incorporating the reagent into the cartridge for cell processing.

The embodiment described above can be appropriately modified and implemented by changing some of the components or functions included in each device. In the following, modifications according to the embodiment above are described as other embodiments. The following mainly describes the points different from the embodiment above and does not describe the points common to the already described contents in detail. The modifications described below may be implemented individually or in combination with the embodiment described above as appropriate.

### First Modification

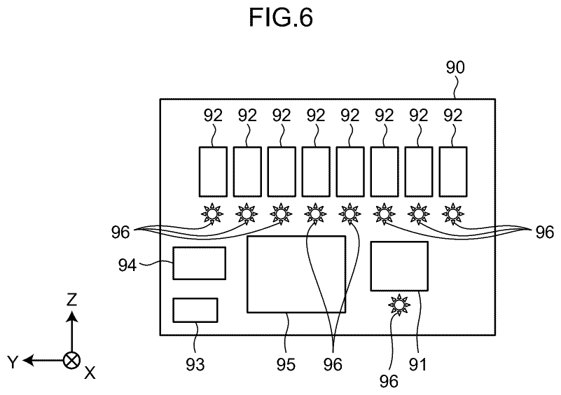

The first outer cover 90, for example, may further include a plurality of notifiers that notify a user of the opening/closing portion corresponding to the connection to which the reagent container is to be connected. The notifiers are described below with reference to FIG. 6.

FIG. 6 is a schematic of an example of the configuration of the first outer cover 90 according to a first modification. The first outer cover 90 illustrated in FIG. 6 further includes a plurality of notifiers 96 compared with the first outer cover 90 illustrated in FIG. 4. The notifier 96 is a light-emitting diode (LED), for example. Each notifier 96 is positioned near the opening/closing portion, and specifically under the opening/closing portion, for example. In other words, the notifiers 96 are provided at the positions corresponding to the respective opening/closing portions.

The notifier 96 notifies the user by emitting light that corresponds to the reagent container to be placed. The notifier 96 is controlled by the acquisition function 841 of the door opening/closing unit 80. Specifically, the acquisition function 841 of the door opening/closing unit 80 controls the notifier 96 such that the notifier 96 operates to emit light that corresponds to the identification information of the reagent container 204, based on the identification information. The notifier 96 emits light.

The notifier 96 notifies the user of the opening/closing portion corresponding to the connection to which the reagent container is to be connected by emitting light. The notifier 96 is controlled by the detection function 842 of the door opening/closing unit 80. Specifically, when the detection function 842 of the door opening/closing unit 80 detects that the opening/closing portion has been opened, the detection function 842 controls the notifier 96 such that the notifier 96 operates to emit light that corresponds to the opening information indicating that the opening/closing portion 91 has been opened. The notifier 96 emits light.

Thus, the operator can find out that the opening/closing portion has been opened by light emission and connect the biological sample container 201 or the reagent container 204 to the connection channel 10 without erroneously connecting them.

Also in the present modification, the processing circuit 84 or its functions may be provided to the cell production device 200. The auxiliary storage device 82 may also be provided to the cell production device 200.

### Second Modification

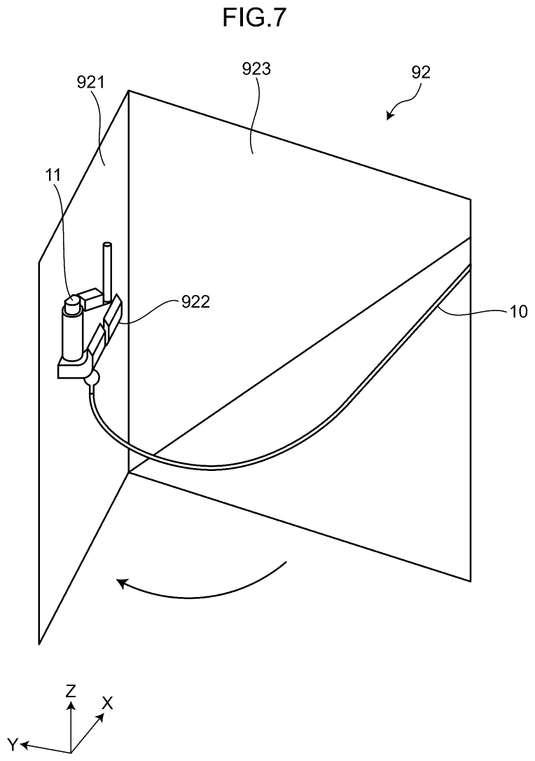

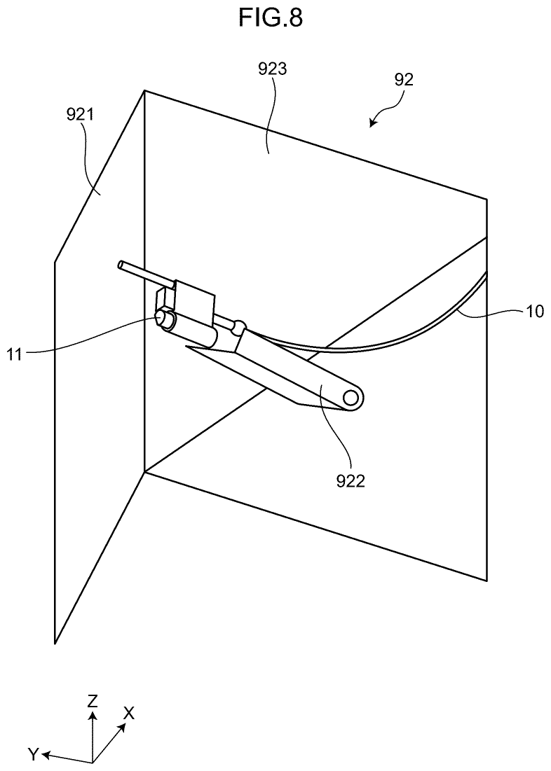

The hinged door of the opening/closing portion 92 may include a holder that holds a connection 11 of the connection channel 10, for example. The holder is described below with reference to FIGS. 7 and 8.

FIGS. 7 and 8 are schematics of an example of the configuration of the opening/closing portion 92 according to a second modification. The opening/closing portion 92 illustrated in FIG. 7 corresponds to the opening/closing portion 92 illustrated in FIG. 4. FIGS. 7 and 8 illustrate the opening/closing portion 92 with a hinged door 921 opened. The opening/closing portion 92 illustrated in FIGS. 7 and 8 includes a holder 922 that holds the connection 11 of the connection channel 10. The holder 922 holds the connection 11 of the connection channel 10 in advance.

FIG. 7 is a schematic of an example of the mechanism in which the holder 922 is provided to the hinged door 921. With this mechanism, when the opening/closing portion 92 is opened, the holder 922 comes closer to the operator as the hinged door 921 rotates, thereby facilitating the operator's accessing the connection 11 held by the holder 922. In other words, the holder 922 moves from the opening of the opening/closing portion 92 so as to face the outside of the cartridge 100 as the opening/closing portion 92 is opened.

The operator removes the connection 11 of the connection channel 10 from the holder 922, connects the connection 11 to the connection of the reagent container, and places the reagent container in a container placement space 923. The operator then closes the hinged door 921 to bring the opening/closing portion 92 into the closed state.

FIG. 8 is a schematic of an example of another mechanism in which the holder 922 is provided on the lower surface of the container placement space 923. The holder 922 is provided on the lower surface of the container placement space 923 with a spring-loaded hinge interposed therebetween, for example, and is biased in the direction of leaning against the hinged door 921.

With this configuration, the holder 922 and the connection 11 held by the holder 922 move from the opening of the opening/closing portion 92 so as to face the outside of the cartridge 100 as the opening/closing portion 92 is opened, and more preferably at least part of the connection 11 moves to protrude from the opening. This mechanism facilitates the operator's accessing the connection 11 held by the holder 922.

The operator removes the connection 11 of the connection channel 10 from the holder 922, connects the connection 11 to the connection of the reagent container, and places the reagent container in the container placement space 923. The operator then closes the hinged door 921 to bring the opening/closing portion 92 into the closed state. Also in the present modification, the processing circuit 84 or its functions may be provided to the cell production device 200. The auxiliary storage device 82 may also be provided to the cell production device 200.

### Third Modification

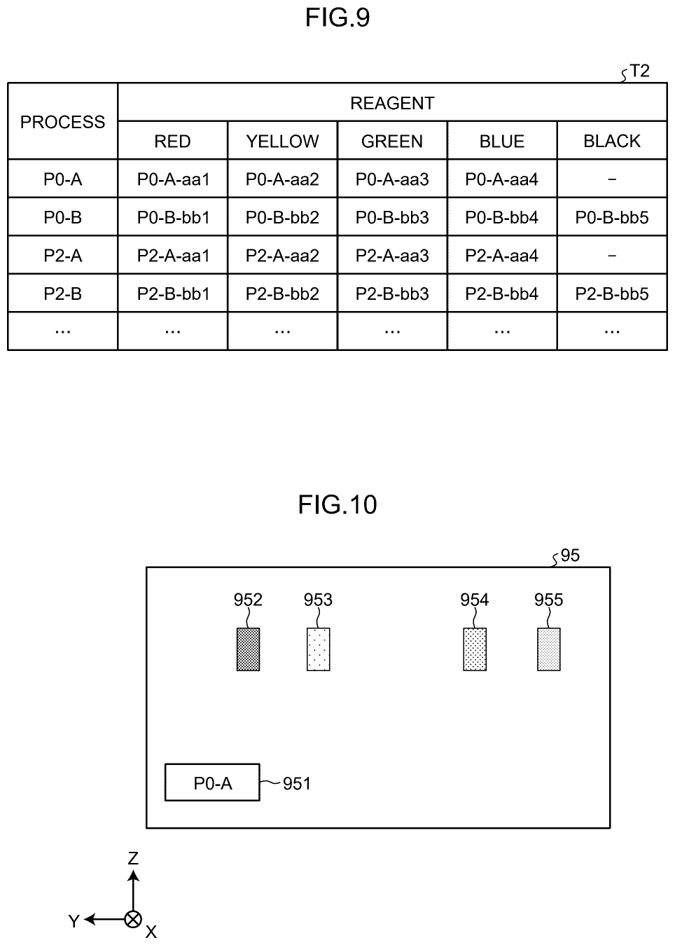

The display unit 95 may output display information indicating the correspondence between the reagent container to be placed in the container placement space 923 and the opening/closing portion, for example. The display information is described below with reference to FIGS. 9 and 10.

FIG. 9 is a table indicating an example of the identification information according to a third modification. Table T2 illustrated in FIG. 9 associates the reagent containers 204 with respective colors compared with Table T1 illustrated in FIG. 5. In other words, the identification information held by the information holder 93 of the cartridge 100 according to the third modification is information including process information indicating a process, such as cell culture, to be performed in the cartridge 100, reagent information indicating reagents used in the process, and color information associating the reagents with colors.

FIG. 10 is a schematic of an example of the display information output by the display unit according to the third modification. The display information displayed by the display unit 95 in FIG. 10 indicates a process 951 and the color information indicating the colors associated with the reagents used in the process out of the identification information in Table T2 illustrated in FIG. 9. The process 951 illustrated in FIG. 10 is text information indicating a process “P0-A”. Colors 952, 953, 954, and 955 of the color information illustrated in FIG. 10 correspond to the process “P0-A”, for example. The color 952 is “red”, the color 953 is “yellow”, the color 954 is “green”, and the color 955 is “blue”.

The control function 843 of the door opening/closing unit 80, for example, controls the display unit 95 such that the display unit 95 displays the text information and the color information that correspond to the identification information of the cartridge 100, based on the identification information. Specifically, the control function 843 indicates the color information on the reagent to be placed on a schematic indicating the position where the reagent is to be placed.

As illustrated in FIG. 10, the display unit 95 displays the text information and the color information at the position indicating the position of the corresponding reagent on a schematic indicating the cartridge 100. Thus, the operator can find out the process of the cartridge 100 and the color information that corresponds to the identification information of the reagent to be placed in each container placement space 923 of the cartridge 100. The display unit 95 is an example of a display unit that indicates the correspondence between the reagent container to be placed and the opening/closing portion. Also in the present modification, the processing circuit 84 or its functions may be provided to the cell production device 200. The auxiliary storage device 82 may also be provided to the cell production device 200.

### Fourth Modification

The cartridge 100 according to the present modification includes a label (process label) indicating information on a process, such as cell culture, to be performed in the cartridge 100 and a label (reagent placement label) indicating the correspondence between the reagent container and the opening/closing portion instead of the display serving as the display unit according to the third modification described above. The process label and the reagent placement label are affixed to the first outer cover 90, for example. The reagent placement label is an example of the display unit that indicates the correspondence between the reagent container to be placed and the opening/closing portion.

The process label displays a text indicating information on the process, such as cell culture, to be performed in the cartridge 100. Thus, the operator can find out at a glance the process to be performed in the cartridge 100 and can recognize the combination of the reagents used in the cartridge 100, for example. A combination of reagents is provided as a kit for each process, for example, and the operator can select the kit for the process by referring to the process label.

The reagent placement label indicates which of the reagents used in the cartridge 100 is to be placed from the opening/closing portion. For example, each reagent is associated with a color in the kit of the combination of the reagents described above, and the reagent placement label of the cartridge 100 indicates the correspondence between the color and the position of the opening/closing portion. Specifically, similarly to the notifiers 96 according to the first modification, for example, the reagent placement labels in colors corresponding to the reagents to be placed are provided to the respective opening/closing portions used to place the reagents. Each reagent placement label is positioned near the opening/closing portion, and specifically under the opening/closing portion, for example.

In the reagent placement label, the correspondence between the color and the position of the opening/closing portion may be indicated by a diagram. In this case, one reagent placement label simply needs to be provided to the first outer cover 90, for example.

While the embodiment and the modifications above have described the cartridge for cell production, the cell production device, and the cell production system for producing iPS cells, for example, the embodiments are not limited thereto. For example, the embodiments described above are also applicable to production of cells other than iPS cells. Furthermore, the embodiments are also applicable not only to production of cells but also to cartridges, devices, and systems that only perform processing on cells. The production of iPS cells or the processing included in the production is an example of “processing of a cell”.

A cartridge for cell processing according to at least one of the embodiments described above can reduce the complexity of incorporating a reagent into the cartridge.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cartridge in which processing of a cell is performed, the cartridge comprising:
a channel through which a reagent used in the processing flows;
a connection configured to connect a reagent container containing the reagent to the channel; and
an opening/closing portion for connecting the reagent container to the connection and placing the reagent container in the cartridge.

2. The cartridge according to claim 1, further comprising an information holder configured to hold identification information for identifying the cartridge.

3. The cartridge according to claim 1, further comprising a processor configured to detect that the opening/closing portion has been opened.

4. The cartridge according to claim 1, further comprising:
a plurality of the opening/closing portions; and
an opening/closing drive mechanism configured to open and close the opening/closing portion and a processor configured to control the opening/closing drive mechanism.

5. The cartridge according to claim 1, wherein
the cartridge is used, being mounted on a cell processing device configured to control execution of the processing of a cell in the cartridge,
the cartridge comprises a plurality of the opening/closing portions, and
the opening/closing portions each comprise an opening/closing drive mechanism configured to open and close the opening/closing portion based on control by the cell processing device.

6. The cartridge according to claim 1, further comprising:
a plurality of the opening/closing portions; and
a plurality of notifiers configured to notify a user of the opening/closing portion corresponding to the connection to which the reagent container is to be connected.

7. The cartridge according to claim 6, wherein the notifiers notify the user of the opening/closing portion corresponding to the connection to which the reagent container is to be connected by emitting light.

8. The cartridge according to claim 7, wherein the notifiers notify the user by emitting light that corresponds to the reagent container to be placed.

9. The cartridge according to claim 1, wherein
the opening/closing portion comprises a hinged door configured to open toward outside of the cartridge, and
the hinged door comprises a holder that holds the connection configured to connect the reagent container.

10. The cartridge according to claim 1, further comprising:
a holder that holds the connection configured to connect the reagent container, wherein
the holder moves from an opening of the opening/closing portion so as to face outside of the cartridge as the opening/closing portion is opened.

11. The cartridge according to claim 1, further comprising:
a plurality of the opening/closing portions; and
a display unit configured to indicate correspondence between the reagent container to be placed and the opening/closing portion.

12. A system comprising the cartridge according to claim 5 and the cell processing device, wherein the cell processing device comprises a processor configured to control the opening/closing drive mechanism of the mounted cartridge.

13. The system according to claim 12, wherein the processor determines an opening/closing portion to be opened out of the opening/closing portions, based on identification information of the reagent.

14. A system comprising the cartridge according to claim 6 and a cell processing device on which the cartridge is mounted, wherein the cell processing device comprises a processor configured to determine a notifier to be caused to make notification out of the notifiers, based on identification information of the reagent.

15. A system comprising the cartridge according to claim 2 and a cell processing device on which the cartridge is mounted, wherein
the information holder of the cartridge includes a radio frequency identification (RFID) tag, and
the cell processing device comprises a processor configured to determine whether an article is lacking based on the RFID tag of the information holder and an RFID tag attached to the reagent container.
